(19) United States
(12) Patent Application Publication
(10) Pub. No.: US 2012/0288554 A1
(43) Pub. Date: Nov. 15, 2012

(54) CANFOSFAMIDE MONOTHERAPY FOR TREATING MULTIPLE MYELOMA

(75) Inventor: Gail L. Brown, Woodside, CA (US)

(73) Assignee: Telik, Inc.

(21) Appl. No.: 13/106,702

(22) Filed: May 12, 2011

(51) Int. Cl. A61K 9/127; C07F 9/24; A61P 35/00; A61K 39/395; A61K 31/675; A61K 31/664; A61K 31/69

(52) U.S. Cl. 424/450; 514/118; 558/171; 514/64; 424/133.1; 514/110

(57) ABSTRACT

Provided herein are methods for treating multiple myeloma by administering canfosfamide or a pharmaceutically acceptable salt thereof as a monotherapy, and also by administering canfosfamide or a pharmaceutically acceptable salt thereof as part of novel combination therapies further comprising one or more of bortezomib, lenalidomide, vorinostat, carfilzomib, pomalidomide, and elotuzumab.

### BACKGROUND OF THE INVENTION

1. Field of Invention

This invention provides methods for treating multiple myeloma with canfosfamide or a pharmaceutically acceptable salt thereof administered as monotherapy, and as part of certain novel combination therapy regimens.

2. State of the Art

Multiple Myeloma is a hematologic malignancy characterized by the proliferation of a single clone of plasma cells engaged in the production of an immunoglobulin. Bone pain, anemia, and fatigue constitute some of the symptoms of multiple myeloma. Hypercalcemia and renal insufficiency are also manifestations of this malignancy.

Conditions associated with a diagnosis of multiple myeloma include bone marrows with greater than 10% plasma cells or plasmacytoma coupled with one or more of the following: monoclonal protein in serum (usually greater than 3 g/deciliter (dL)), monoclonal protein in urine, and lytic bone lesions. Multiple myeloma accounts for more than 10% of hematologic malignancies with the incidence of approximately of 1 to 4 individuals per 100,000 per year. For multiple myeloma, the median age at diagnosis is 61; the advanced age itself limits the types of treatment the patient can undergo.

Chemotherapy is the preferred initial treatment for multiple myeloma. Yet, for chemotherapy alone, the 5 year survival probability is only 12%. Almost all patients with multiple myeloma who respond positively to chemotherapy will eventually experience relapse. Patients relapse in a large part because their multiple myeloma becomes resistant or refractory to the initial treatment, and such patients are then preferably treated with a combination regimen of drugs. For example, patients with multiple myeloma resistant to alkylating agents are treated with a combination regimen called VAD (vincristine, doxorubicin or adriamycin, and dexamethasone).

As part of a chemotherapy regimen, thalidomide has been evaluated in relapsed/refractory multiple myeloma patients, though thalidomide combinations with chemotherapy, specifically anthracyclines, carry an increased risk of venous thromboembolic (VTE) complications, which often require extensive patient monitoring and intense prophylaxis. Thalidomide alone produced partial remission rates in about 30% of patients. The overall response rate can be enhanced when thalidomide is administered as part of a drug combination; however the improvement in response rate comes with an increase in the undesirable side effects, such as VTE, as discussed above.

Bortezomib is a proteasome inhibitor with antimyeloma activity as a single agent, though bortezomib-induced peripheral neuropathy remains a dose-limiting toxicity in patients with multiple myeloma, which often requires adjustment of treatment and affects quality of life.

Lenalidomide is an agent approved recently for relapse/refractory myeloma in the United States and Europe. However, as with its structural analog thalidomide, lenalidomide is associated with adverse effects related to VTE. Nearly all lenalidomide trials show an increased risk of VTE with lenalidomide therapy, with the risk significantly increasing when lenalidomide is combined with dexamethasone or with other combination chemotherapy.

Autologous stem cell transplantation is useful for up to 50% of multiple myeloma patients who are candidates for such transplantation. Despite a low mortality rate, problems with such transplant therapy include the inability to eradicate the tumor and the difficulty in the removal of myeloma cells and their precursors from the stem cell collection used for transplantation.

Allogenic transplant is another therapy option for treating multiple myeloma, but is less frequently used since the mortality rate at 100 days is 25-30% and it does not provide a cure. Only 5-10% of patients with multiple myeloma are eligible for allogenic bone marrow transplantation because of their age and the paucity of a human leukocyte antigen (HLA)-matched sibling donor.

The use of allogenic transplant for the treatment of refractory and/or relapsed myeloma also remains a treatment strategy with limited clinical benefit. Most studies evaluating its use in this setting demonstrate long-term disease-free survival of 10-20%, with a significant fraction of patients developing debilitating chronic graft versus host disease or relapse. Given the significant limitations of treatment-related mortality, morbidity, and poor overall outcomes, the use of allogenic transplant for the management of relapsed myeloma is deemed ineffective.

As discussed above, conventional multiple myeloma treatment often involves administering a combination of drugs or therapies, typically referred to as combination therapy. As an example of such a combination therapy, the use of canfosfamide hydrochloride, in combination with other chemotherapy agents, has been contemplated for treating multiple myeloma. See, e.g., U.S. App. Pub. No. 2004/0138140.

In spite of the prevalence of combination therapies for treating multiple myeloma, combination therapies can be problematic, as discussed above, due to their resulting toxic side effects. Because of the interplay of many toxic side effects, the overall toxic effects of a combination therapy may be heightened compared to the toxic side effects of the individual therapies or drugs employed in it.

Notwithstanding the drawbacks of combination therapy, monotherapy is considered ineffective for treating patients with multiple myeloma especially with refractory and/or relapsed multiple myeloma. The reduced or lack of efficacy of monotherapy results in part from the fact that it is easier for the cancer cells to be resistant to the single agent or therapy employed to kill such cells.

There is a need for a new treatment regimen for multiple myeloma involving monotherapy with a single active agent, particularly for patients who can not undergo stem cell transplantation. At the same time, it is surprising and unexpected that a monotherapy employing a single active agent may be effective in treating multiple myeloma especially if it has become refractory to other conventional drugs and/or has relapsed.

### SUMMARY OF THE INVENTION

In one aspect, this invention provides a method of treating multiple myeloma comprising administering a therapeutically effective amount of canfosfamide or a pharmaceutically acceptable salt thereof as a monotherapy to a patient in need of such treatment. In one embodiment, the pharmaceutically acceptable salt is canfosfamide hydrochloride. In one embodiment, multiple myeloma that is refractory to a variety of chemotherapy and/or other therapies is treated according to this invention. In another embodiment, multiple myeloma that has relapsed after previous therapy is also treated according to this invention.

### DETAILED DESCRIPTION OF THE INVENTION

As noted above, this invention is directed, in part, to treating multiple myeloma by administering canfosfamide hydrochloride as monotherapy. However, prior to discussing this invention in further detail, the following terms will be defined.

### DEFINITIONS

As used herein, the following terms have the following meanings

The singular forms “a,” “an,” and “the” and the like include plural referents unless the context clearly dictates otherwise. Thus, for example, reference to “a drug” includes both a single drug and a plurality of different drugs.

The term “about” when used before a numerical designation, e.g., temperature, time, amount, and concentration, including a range, indicates approximations which may vary by ±10%, ±5, or ±1%.

“Administration” refers to introducing an agent into a patient. A therapeutic amount can be administered, which can be determined by the treating physician or the like. A parenteral route of administration is preferred for monotherapy according to this invention. The related terms and phrases “administering” and “administration of”, when used in connection with a compound or pharmaceutical composition (and grammatical equivalents) refer both to direct administration, which may be administration to a patient by a medical professional and/or to indirect administration, which may be the act of prescribing a drug. For example, a physician who provides a patient with a prescription for a drug is administering the drug to the patient. In any event, administration entails delivery to the patient of the drug.

“Comprising” or “comprises” is intended to mean that the methods include the recited elements, but do not exclude others. “Consisting essentially of” when used to define methods, shall mean excluding other elements of any essential significance to the combination for the stated purpose. Thus, a method consisting essentially of the elements as defined herein would not exclude other steps that do not materially affect the basic and novel characteristic(s) of the claimed invention. “Consisting of” shall mean excluding substantial other method steps. Embodiments defined by each of these transition terms are within the scope of this invention.

“Monotherapy” refers to administering a single active agent for treating a condition, such as multiple myeloma.

“Multiple Myeloma” refers to a hematologic malignancy characterized by a proliferation of a single clone of plasma cells engaged in the production of an immunoglobulin. Bone pain, anemia, and fatigue constitute certain symptoms of multiple myeloma. Hypercalcemia and renal insufficiency are important manifestations of this hematologic malignancy. Conditions associated with a diagnosis of multiple myeloma include, without limitation, bone marrows with greater than 10% plasma cells or plasmacytoma coupled with one or more of the following: monoclonal protein in serum (usually greater than 3 g/deciliter (dL)), monoclonal protein in urine, and lytic bone lesions.

As used herein, “refractory and/or relapsed multiple myeloma” is refractory to one or more of chemotherapy, and/or resistant to one or more of chemotherapy or other therapy, and/or relapsed after treatment with one or more of chemotherapy or other therapy, where the chemotherapy includes, without limitation, monotherapy and combination therapy involving cyclophosphamide, dexamethasone, doxorubicin, etoposide, interferon-alpha, melphalan, pegylated interferon-alpha, vincristine, and the like, corticosteroids, such as prednisone, dexamethasone (e.g., decadron), and the like, and immune modulating agents such as thalidomide, lenalidomide (Revlimid®, Celgene), and bortezomib (Velcade®, Millennium Pharmaceuticals), and the like. Various combinations of such agents intended for treating multiple myeloma is well known to the skilled artisan. For example, and without limitation, such combination chemotherapy include revlimid/melphalan/prednisone, revlimid/decadron, velcade/decadron, velcade/revlimid/decadron, and EPOCH (etoposide, prednisone, vincristine (oncovin), doxorubicin, and cyclophosphamide). As used herein, “other therapy” includes, without limitation, radiation therapy and autologous stem cell transplant therapy. For example, and without limitation, such radiation therapy is preferably administered to the humerus, thoracic/lumbar spine and/or the sacrum.

“Patient” refers to a human patient.

As used herein, patients “ineligible to undergo autologous stem cell transplantation” includes, without limitation, patients with renal failure, who are contemplated to be dialysis-dependent, and patients who are too old to undergo such stem cell transplantation.

“Refractory and/or relapsed multiple myeloma” refers to a multiple myeloma unresponsive to a drug (a chemotherapy agent) and/or another therapy administered prior to treatment with canfosfamide or a pharmaceutically acceptable salt thereof and includes multiple myeloma resistant to one or more of such prior administered chemotherapy or other therapy. For example and without limitation, refractory and/or relapsed multiple myeloma includes multiple myeloma in patients whose first progression occurs in the absence of any treatment following successful treatment with a drug or a therapy; multiple myeloma in patients who progress on a treatment, or within 60 days of the treatment; and multiple myeloma in patients who progress while receiving treatment. Examples of refractory and/or relapsed multiple myeloma include, without limitation, bortezomib refractory relapse or lenalidomide refractory relapse multiple myeloma.

“Single active agent” refers to an agent that is useful for treating a condition, such as multiple myeloma, when administered without coadministering during the course of the treatment with the single active agent, one or more chemotherapy agents or, preferably, one or more other therapies suitable for treating multiple myeloma. Thus, a single active agent, such as canfosfamide hydrochloride, is effective in treating multiple myeloma, without coadministering a chemotherapy agent or, preferably, stem cell transplantation or radiation therapy.

“Therapeutically effective amount” or “therapeutic amount” refers to an amount of a drug or an agent that when administered to a patient suffering from a condition, will have the intended therapeutic effect, e.g., alleviation, amelioration, palliation or elimination of one or more symptoms or manifestations of the condition in the patient. For example, and without limitation, when the condition treated is multiple myeloma, the relevant symptoms or manifestations include, one or more of, proliferation of a single clone of plasma cells engaged in the production of a specific immunoglobulin, a bone marrow with >10% plasma cells or plasmacytoma, monoclonal protein in serum (preferably >3 g/dL), monoclonal protein in urine, lytic bone lesions or bone pain, anemia, fatigue, hypercalcemia, and renal insufficiency. The therapeutically effective amount will vary depending upon the subject and the condition being treated, the weight and age of the subject, the severity of the condition, the particular composition or excipient chosen, the dosing regimen to be followed, timing of administration, the manner of administration and the like, all of which can be determined readily by one of ordinary skill in the art. The full therapeutic effect does not necessarily occur by administration of one dose, and may occur only after administration of a series of doses. Thus, a therapeutically effective amount may be administered in one or more administrations. For example, and without limitation, a therapeutically effective amount of an agent, such as canfosfamide, in the context of treating multiple myeloma, refers to an amount of the agent that alleviates, ameliorates, palliates, or eliminates one or more manifestations of the multiple myeloma in the patient. It is preferred that the therapeutically effective amount of the agent is an amount that is administered as monotherapy, though, for patients showing symptoms of tumor lysis syndrome, the agent is contemplated to be administered in combination with an agent that is useful in inhibiting or treating the tumor lysis syndrome, and it is also contemplated that the therapeutically effective amount is administered as part of a combination therapy comprising canfosfamide or a pharmaceutically acceptable salt thereof, and one or more of bortezomib, lenalidomide, vorinostat, carfilzomib, pomalidomide, and elotuzumab.

“Treatment”, “treating”, and “treat” are defined as acting upon a disease, disorder, or condition with an agent to reduce or ameliorate the harmful or any other undesired effects of the disease, disorder, or condition and/or its symptoms. Treatment, as used herein, covers the treatment of a human patient, and includes: (a) reducing the risk of occurrence of the condition in a patient determined to be predisposed to the disease but not yet diagnosed as having the condition, (b) impeding the development of the condition, and/or (c) relieving the condition, i.e., causing regression of the condition and/or relieving one or more symptoms or manifestations of the condition. For example, and without limitation, when the condition treated is multiple myeloma, the relevant symptoms or manifestations include, without limitation, proliferation of a single clone of plasma cells engaged in the production of a specific immunoglobulin, a bone marrow with >10% plasma cells or plasmacytoma, monoclonal protein in serum (preferably >3 g/dL), monoclonal protein in urine, lytic bone lesions or bone pain, anemia, fatigue, hypercalcemia, and renal insufficiency. It is preferred that the disease treated is multiple myeloma, though treatment of tumor lysis syndrome related to multiple myeloma treatment is also contemplated.

“Tumor lysis syndrome” (TLS) refers to a group of metabolic complications that can occur after treatment of a hematologic malignancy, such as multiple myeloma. These complications are caused by the products of the dying malignant cells and include as symptoms hyperkalemia, hyperphosphatemia, hyperuricemia, hyperuricosuria, hypocalcemia, acute uric acid nephropathy, or acute renal failure. Agents and therapies useful for treating TLS include allopurinol, loop diuretics, rasburicase, and/or hemodialysis. TLS may be inhibited or prevented by pretreatment with allopurinol or rasburicase.

### Treatment of Multiple Myeloma

In one aspect, this invention provides a method of treating multiple myeloma comprising administering a therapeutically effective amount of canfosfamide or a pharmaceutically acceptable salt thereof as a monotherapy to a patient in need of such treatment. In one embodiment, the pharmaceutically acceptable salt of canfosfamide is canfosfamide hydrochloride. In another embodiment, the multiple myeloma is refractory and/or relapsed multiple myeloma. In another embodiment, the multiple myeloma is refractory to a variety of chemotherapy and/or other therapies. In another embodiment, the multiple myeloma relapsed after previous therapy. In another embodiment, the multiple myeloma relapsed after treatment with one or more rounds of chemotherapy. In another embodiment, the multiple myeloma relapsed after autologous or allogenic stem cell transplantations. In another embodiment, the patient is ineligible to undergo autologous stem cell transplantation. Autologous stem cell transplantation is preferably not used for patients over the age of 65 years, particularly if they have substantial heart, lung, renal or liver dysfunction.

In another embodiment, the therapeutically effective amount is about 500 mg/m²/day-about 1000 mg/m²/day. In another embodiment, the canfosfamide hydrochloride is administered once every week, once every two weeks, or once every three weeks. Once a week administration is contemplated for the lower end of the dosing range, e.g., for administering a dose of about 500 mg/m²/day. With reduced dosing frequency, larger amounts are contemplated to be dosed, such as administering a dose of about 1000 mg/m²/day for a dosing frequency of once every two weeks or once every three weeks. In another embodiment, the canfosfamide hydrochloride is administered by intravenous infusion. In another embodiment, the infusion is performed for about 30 minutes. In another embodiment, if the patient shows symptoms of tumor lysis syndrome, the method further comprises administering an agent or a therapy for inhibiting or treating the tumor lysis syndrome.

Tumor lysis syndrome is not very commonly observed for multiple myeloma patients undergoing chemotherapy. However, in rare instances associated with the administration of bortezomib and thalidomide, agents that are approved for treating multiple myeloma, occurrence of tumor lysis syndrome in multiple myeloma patients were reported. Tumor lysis syndrome was observed, after the first cycle of canfosfamide hydrochloride administration according to this invention. Based on such an unexpected effect, it is contemplated that canfosfamide hydrochloride demonstrates surprising single agent efficacy for treating multiple myeloma. When a patient starts showing early symptoms of tumor lysis syndrome, the syndrome can be inhibited and/or treated with various known therapies, including, without limitation, those involving allopurinol and/or hydration.

Based partly on the positive monotherapy outcomes observed for administering canfosfamide hydrochloride to multiple myeloma patients, in another aspect of this invention, it is contemplated that novel combinations of canfosfamide or a pharmaceutically acceptable salt thereof and another drug suitable for treating multiple myeloma can be advantageously administered to treat multiple myeloma. Such combinations comprise a therapeutically effective amount of canfosfamide or a pharmaceutically acceptable salt thereof in combination with one or more of bortezomib, lenalidomide, vorinostat, carfilzomib, pomalidomide, and elotuzumab. In certain embodiments, the novel combinations provided here are administered to a patient in need of multiple myeloma treatment. As used herein, administration in “combination” or “coadministration” refers to the administration of the two or more drugs (e.g., canfosfamide or a pharmaceutically acceptable salt thereof and one or more additional drugs to treat multiple myeloma) in any manner in which the pharmacological effects of both are manifest in the patient at the same time. Thus, administration in combination does not require that a single pharmaceutical composition, the same dosage form, or even the same route of administration be used for administration of both canfosfamide or a pharmaceutically acceptable salt thereof and the additional drug(s) to treat multiple myeloma, or that the drugs be administered at precisely the same time. However, administration in combination is contemplated to preferably be accomplished by the same dosage form and the same route of administration, at substantially the same time.

In certain embodiments, the combination comprises administering a binary composition of canfosfamide or a pharmaceutically acceptable salt thereof and bortezomib, or alternatively, administering ternary or higher compositions including canfosfamide or a pharmaceutically acceptable salt thereof and bortezomib. Non-limiting examples of such ternary or higher compositions include canfosfamide or a pharmaceutically acceptable salt thereof and:

(a) bortezomib/thalidomide/dexamethasone;
(b) bortezomib/pegylated liposomal doxorubicin (PLD)/dexamethasone;
(c) bortezomib/PLD/thalidomide;
(d) bortezomib/PLD;

### (e) PLD/bortezomib/thalidomide/dexamethasone;

(f) bortezomib/dexamethasone/cyclophosphamide;
(g) bortezomib/cyclophosphamide/thalidomide/dexamethasone;
(h) bortezomib/cyclophophamide/predisone;
(i) bortezomib/dexamethasone/cyclophosphamide;
(j) bortezomib/doxorubicin/dexamethasone;
(k) bortezomib/doxorubicin/dexamethasone then thalidomide/dexamethasone;
(l) bortezomib/melphalan/dexamethasone;
(m) bortezomib/melphalan/dexamethasone/intermittent thalidomide;
(n) bortezomib/melphalan/prednisone/intermittent thalidomide;
(o) bortezomib/melphalan;
(p) vorinostat/bortezomib;
(q) panobinostat/bortezomib; or
(r) elotuzumab/bortezomib.

In certain embodiments, the combination comprises administering a binary composition of canfosfamide or a pharmaceutically acceptable salt thereof and lenalidomide, or alternatively, administering ternary or higher compositions including canfosfamide or a pharmaceutically acceptable salt thereof and lenalidomide. Non-limiting examples of such ternary or higher compositions include canfosfamide or a pharmaceutically acceptable salt thereof and:

(a) lenalidomide/dexamethasone;
(b) lenalidomide/PLD/dexamethasone;
(c) cyclophosphamide/leanlidomide/dexamethasone;
(d) lenalidomide/adriamycin/dexamethasone;
(e) lenalidomide/melphalan/prednisone/thalidomide;
(f) vorinostat/lenalidomide/dexamethsone;
(g) panobinostat/lenalidomide/dexamethasone; or
(h) elotuzumab/lenalidomide/dexamethasone.

The therapy comprising bortezomib, revlimid, vorinostat, carfilzomib, pomalidomide, or elotuzumab is contemplated to be administered following the regimens known for administering such therapies.

In one embodiment, this invention provides a pharmaceutical composition for use in the treatment of multiple myeloma, wherein the pharmaceutical composition comprises a therapeutically effective amount of canfosfamide or a pharmaceutically acceptable salt thereof as a single active agent. In another embodiment the pharmaceutical composition comprises a therapeutically effective amount of canfosfamide or a pharmaceutically acceptable salt thereof and one or more of bortezomib, lenalidomide, vorinostat, carfilzomib, pomalidomide, and elotuzumab. In another embodiment, the multiple myeloma is refractory and/or relapsed multiple myeloma.

In one embodiment, this invention provides the use of canfosfamide or a pharmaceutically acceptable salt thereof as a single active agent for the manufacture of a medicament for the treatment of multiple myeloma. In another embodiment, the medicament further comprises one or more of bortezomib, lenalidomide, vorinostat, carfilzomib, pomalidomide, and elotuzumab. In another embodiment, the multiple myeloma is refractory and/or relapsed multiple myeloma.

For administration in accordance with this invention, canfosfamide hydrochloride is formulated as a lyophilized solid. Lyophilized canfosfamide hydrochloride and methods of preparing it is described in U.S. patent application Ser. No. 13/035,637, filed on Feb. 25, 2011, entitled “FORMULATIONS OF CANFOSFAMIDE AND THEIR PREPARATION,” which is incorporated herein by reference in its entirety.

The invention having been discussed in summary and in detail, is illustrated and not limited by the following example

### Example

### Administration of Canfosfamide Hydrochloride as a Monotherapy for Treatment of Multiple Myeloma in Human Patients

This example is a single arm, open label, Phase 2 clinical test that tests the safety and efficacy of canfosfamide monotherapy for the treatment of refractory or relapsed multiple myeloma. Refractory or relapsed multiple myeloma patients were administered canfosfamide hydrochloride in an amount of 1000 mg/m², intravenously (IV), over 30 minutes, once every 2 weeks (a cycle of therapy). Response was assessed after every 3 cycles of therapy. Of the three multiple myeloma patients treated, two patients, who had high risk multiple myeloma refractory to several prior combination chemotherapies and other therapies, demonstrated positive treatment outcomes and were evaluable for response as tabulated below.

| | Patient No. 1 | Patient No. 2 |
|---|---|---|
| Age and sex | 83 year old male | 59 year old male |
| Durie Salmon stage | IIIA | IIIA |
| International staging | ISS stage III | ISS stage III |
| system (ISS) stage | | |
| Eastern Cooperative | 2 | 2 |
| Oncology Group (ECOG) | | |
| performance status | | |
| Previous chemotherapy | Radiation to humerus | Combination chemotherapy with |
| and other therapy | and thoracic/lumbar spine and | velcade/decadron, radiation to |
| | combination chemotherapy with | lumbar/sacrum, and further |
| | revlimid/melphalan/prednisone | combination chemotherapy with |
| | and revlimid/decadron. | velcade/revlimid/decadron, with |
| | | EPOCH (etoposide, prednisone, |
| | | vincristine (oncovin), doxorubicin, |
| | | and cyclophosphamide), and |
| | | autologous stem cell |
| | | transplantation. |
| Baseline disease values | Serum protein electrophoresis | SPEP M-spike: 5.37 g/dL |
| | monoclonal (M) protein level | Kappa FLC:<0.11 mg/dL |
| | (SPEP M-spike): 1.3 g/dL | Lambda FLC: 282 mg/dL |
| | Kappa monoclonal free light | FLC Ratio: unable to calculate |
| | chains (FLC): 3.94 mg/dL | 24 hour UPEP: 7.9 grams. |
| | Lambda FLC: 0.19 mg/dL | Bone Marrow biopsy: 80% |
| | FLC Ratio (kappa to lambda): | plasma cells. |
| | 20.74 | FISH detects del13q and 1q |
| | 24 hour urine protein | abnormality. Cytogenetics |
| | electrophoresis (UPEP): 0.4 | demonstrates complex female |
| | grams with only IFE positive | karyotype including del13q. |
| | Bone marrow biopsy: 75% | |
| | plasma cells. | |
| | Fluorescence in situ | |
| | hybridization (FISH) detects 1q | |
| | abnormality. | |
| Disease response after | SPEP M-spike: 1.0 g/dL | SPEP M-spike: 3.18 g/dL |
| cycle #3 of monotherapy | Kappa FLC: 1.66 mg/dL | Kappa FLC: <0.11 mg/dL |
| | Lambda FLC: 0.23 mg/dL | Lambda FLC: 218 mg/dL |
| | FLC Ratio: 7.22 | FLC Ratio: unable to calculate |
| | 24 hour UPEP: 0.2 grams with | 24 hour UPEP: 5.8 grams. |
| | only IFE positive. | Overall, the patient exhibited |
| | Overall, the patient exhibited | “partial response,” which may be |
| | “minimal response,” which may | characterized, e.g., by about 50% |
| | be characterized, e.g., by about | reduction of serum M-protein. |
| | 25% reduction in serum | |
| | monoclonal paraprotein. | |
| Disease response after | SPEP M-spike: 0.18 g/dL | Currently on cycle 4 of therapy. |
| cycle #6 of monotherapy | Kappa FLC: 1.44 mg/dL | |
| | Lambda FLC: 0.62 mg/dL | |
| | FLC Ratio: 2.32 | |
| | 24 hour UPEP: 0.5 grams with | |
| | albumin and IFE: negative. | |
| | Bone Marrow Biopsy: 20% | |
| | plasma cells. | |
| | FISH detects 1q. | |
| | Overall, the patient exhibited | |
| | “partial response.” | |

When evaluated 6 weeks and 12 weeks after initiating treatment with canfosfamide hydrochloride as monotherapy, patient no. 1 continued to respond to the monotherapy.

This example demonstrates the surprising efficacy of a canfosfamide salt, canfosfamide hydrochloride, administered as a monotherapy, for treating multiple myeloma in human patients. Canfosfamide hydrochloride was effective as monotherapy even when the multiple myeloma treated was refractory to a variety of combination chemotherapy and/or radiation or stem cell transplantation therapy regimens. It is surprising that multiple myeloma that is refractory to a variety of combination treatment regimens responded when treated with a single active agent—canfosfamide hydrochloride. Canfosfamide monotherapy was also effective in treating a patient, who, due to his advanced age, was unable to undergo autologous stem cell transplantation.

While this invention has been described in conjunction with specific embodiments and examples, it will be apparent to a person of ordinary skill in the art, having regard to that skill and this disclosure, that equivalents of the specifically disclosed materials and processes will also be applicable to this invention; and such equivalents are intended to be included within the following claims.

1. A method of treating multiple myeloma comprising administering a therapeutically effective amount of canfosfamide or a pharmaceutically acceptable salt thereof as a monotherapy to a patient in need of such treatment.

2. The method of claim 1, wherein the multiple myeloma is refractory and/or relapsed multiple myeloma.

3. The method of claim 1, wherein the patient is ineligible to undergo autologous stem cell transplantation.

4. The method of claim 1, wherein the therapeutically effective amount is about 500 mg/m²/day-about 1000 mg/m²/day.

5. The method of claim 1, wherein the canfosfamide or the pharmaceutically acceptable salt thereof is administered once every week, once every two weeks, or once every three weeks.

6. The method of claim 1, wherein the canfosfamide or the pharmaceutically acceptable salt thereof is administered by intravenous infusion.

7. The method of claim 6, wherein the infusion is performed for about 30 minutes.

8. The method of claim 1, wherein if the patient shows symptoms of tumor lysis syndrome, further administering an agent or a therapy for inhibiting and/or treating the tumor lysis syndrome.

9. A pharmaceutical composition for use in the treatment of multiple myeloma, wherein the pharmaceutical composition comprises a therapeutically effective amount of canfosfamide or a pharmaceutically acceptable salt thereof as a single active agent.

10. Use of canfosfamide or a pharmaceutically acceptable salt thereof as a single active agent for the manufacture of a medicament for the treatment of multiple myeloma.

11. The pharmaceutical composition of claim 9, wherein the multiple myeloma is refractory and/or relapsed multiple myeloma.

12. The use of claim 10, wherein the multiple myeloma is refractory and/or relapsed multiple myeloma.

13. A method of treating multiple myeloma comprising administering to a patient in need of such treatment a therapeutically effective amount of canfosfamide or a pharmaceutically acceptable salt thereof in combination with bortezomib or a bortezomib therapy selected from the group consisting of:
(a) bortezomib/thalidomide/dexamethasone;
(b) bortezomib/pegylated liposomal doxorubicin (PLD)/dexamethasone;
(c) bortezomib/PLD/thalidomide;
(d) bortezomib/PLD;
(e) PLD/bortezomib/thalidomide/dexamethasone;
(f) bortezomib/dexamethasone/cyclophosphamide;
(g) bortezomib/cyclophosphamide/thalidomide/dexamethasone;
(h) bortezomib/cyclophophamide/predisone;
(i) bortezomib/dexamethasone/cyclophosphamide;
(j) bortezomib/doxorubicin/dexamethasone;
(k) bortezomib/doxorubicin/dexamethasone then thalidomide/dexamethasone;
(l) bortezomib/melphalan/dexamethasone;
(m) bortezomib/melphalan/dexamethasone/intermittent thalidomide;
(n) bortezomib/melphalan/prednisone/intermittent thalidomide;
(o) bortezomib/melphalan;
(p) vorinostat/bortezomib;
(q) panobinostat/bortezomib; and
(r) elotuzumab/bortezomib.

14. A method of treating multiple myeloma comprising administering to a patient in need of such treatment a therapeutically effective amount of canfosfamide or a pharmaceutically acceptable salt thereof in combination with lenalidomide or a lenalidomide therapy selected from the group consisting of:
(a) lenalidomide/dexamethasone;
(b) lenalidomide/PLD/dexamethasone;
(c) cyclophosphamide/leanlidomide/dexamethasone;
(d) lenalidomide/adriamycin/dexamethasone;
(e) lenalidomide/melphalan/prednisone/thalidomide;
(f) vorinostat/lenalidomide/dexamethsone;
(g) panobinostat/lenalidomide/dexamethasone; and
(h) elotuzumab/lenalidomide/dexamethasone.

15. The method of claim 13 or 15, wherein the multiple myeloma is refractory and/or relapsed multiple myeloma.

16. A pharmaceutical composition for use in the treatment of multiple myeloma, wherein the pharmaceutical composition comprises a therapeutically effective amount of canfosfamide or a pharmaceutically acceptable salt thereof and bortezomib or a bortezomib composition selected from the group consisting of:
(a) bortezomib/thalidomide/dexamethasone;
(b) bortezomib/pegylated liposomal doxorubicin (PLD)/dexamethasone;
(c) bortezomib/PLD/thalidomide;
(d) bortezomib/PLD;
(e) PLD/bortezomib/thalidomide/dexamethasone;
(f) bortezomib/dexamethasone/cyclophosphamide;
(g) bortezomib/cyclophosphamide/thalidomide/dexamethasone;
(h) bortezomib/cyclophophamide/predisone;
(i) bortezomib/dexamethasone/cyclophosphamide;
(j) bortezomib/doxorubicin/dexamethasone;
(k) bortezomib/doxorubicin/dexamethasone then thalidomide/dexamethasone;
(l) bortezomib/melphalan/dexamethasone;
(m) bortezomib/melphalan/dexamethasone/intermittent thalidomide;
(n) bortezomib/melphalan/prednisone/intermittent thalidomide;
(o) bortezomib/melphalan;
(p) vorinostat/bortezomib;
(q) panobinostat/bortezomib; and
(r) elotuzumab/bortezomib.

17. A pharmaceutical composition for use in the treatment of multiple myeloma, wherein the pharmaceutical composition comprises a therapeutically effective amount of canfosfamide or a pharmaceutically acceptable salt thereof and lenalidomide or a lenalidomide composition selected from the group consisting of:
(a) lenalidomide/dexamethasone;
(b) lenalidomide/PLD/dexamethasone;
(c) cyclophosphamide/leanlidomide/dexamethasone;
(d) lenalidomide/adriamycin/dexamethasone;
(e) lenalidomide/melphalan/prednisone/thalidomide;
(f) vorinostat/lenalidomide/dexamethsone;
(g) panobinostat/lenalidomide/dexamethasone; and
(h) elotuzumab/lenalidomide/dexamethasone.

18. The pharmaceutical composition of claim 16 or 17, wherein the multiple myeloma is refractory and/or relapsed multiple myeloma.
